(19) United States
(12) Patent Application Publication
(10) Pub. No.: US 2024/0047083 A1
(43) Pub. Date: Feb. 8, 2024

(54) LIBRARIES OF DATA THAT ENABLE PRODUCTION OF PANDEMIC-READY VACCINES AND METHODS OF PREPARING THE SAME

(71) Applicant: Seqirus, Inc., Holly Springs, NC (US)

(72) Inventor: Ethan SETTEMBRE, Cambridge, MA (US)

(21) Appl. No.: 18/264,456

(22) PCT Filed: Feb. 9, 2022
(86) PCT No.: PCT/IB2022/051176

§ 371 Date: Aug. 7, 2023

Related U.S. Application Data
(60) Provisional application No. 63/147,894, filed on Feb. 10, 2021.

(51) Int. Cl. G16H 70/40; G16B 15/30; A61K 39/215

(52) U.S. Cl. CPC G16H 70/40; G16B 15/30; A61K 39/215

(57) ABSTRACT

The present disclosure relates to libraries and stockpiles of data that enable accelerated production of pandemic-ready vaccines.

### FIELD

This invention is in the field of preparedness for pathogens with pandemic potential. The present disclosure relates to libraries of vaccines and data that enable accelerated production of pandemic-ready vaccines and methods of preparing such libraries.

### BACKGROUND

Pandemics remain a constant public health concern. As evidenced by the current COVID-19 pandemic, at any point, a small community pathogenic outbreak has the potential to rise to a global pandemic that causes significant strain on both the health and economy of the world's population. Thus, there is a frequent need for methods to combat the potential for global pandemics.

It is an object of the invention to provide libraries or stockpiles of preclinical and clinical data that allow for rapid development of vaccines in anticipation of pandemics and methods of creating such libraries or stockpiles.

### SUMMARY

This invention generally relates to libraries or stockpiles of preclinical and clinical data that enable rapid development of pandemic-ready vaccines. By way of example, the pathogens may be viruses, bacteria, parasites, or fungi. Exemplary vaccines may be DNA-based, RNA-based, protein-based, or vector-based vaccines.

### DESCRIPTION OF DRAWINGS

FIG. 1 depicts a flowchart and timeline for pandemic packages that accelerate responses to potential pandemics.

FIG. 2 depicts a scheme for preparing self-amplifying mRNA vaccines for pandemic responses.

FIG. 3 depicts a graph demonstrating the differences in levels of CD4⁺ and CD8⁺ T cells following vaccination against COVID-19 with self-amplifying mRNA (blue) or non-amplifying mRNA (orange).

FIG. 4 depicts an image demonstrating the differences when injecting self-amplifying versus non-amplifying mRNA into patients.

FIG. 5 depicts self-amplifying mRNA bicistronic constructs comprising neuraminidase (NA) and hemagglutinin (HA).

FIG. 6 depicts antibody titers in mice against hemagglutinin (H5), neuraminidase (N1), and H5N1 following administration of self-amplifying mRNA bicistronic constructs comprising neuraminidase (NA) and hemagglutinin (HA).

FIG. 7 depicts results of a luciferase bioluminescence assay comparing expression of self-amplifying mRNA (red) versus non-amplifying mRNA (black) post-vaccination.

FIG. 8 depicts (A) a vaccination timeframe for COVID-19-challenged hamsters treated with placebo (PBS) (gray), 3 μg of self-amplifying mRNA (blue), 0.3 μg of self-amplifying mRNA (green), or 5 μg of non-amplifying mRNA+MF59 adjuvant (purple); (B) a graph demonstrating viral load in the nasal turbinates and lungs of COVID-19-challenged hamsters treated with each of the above therapies after four days of infection; and (C) weight relative to pre-infection in COVID-19-challenged hamsters treated with each of the above therapies.

FIG. 9 depicts a flowchart for achieving full authorization of a vaccine in a pandemic when employing data from a library of preclinical and clinical data related to a prototype pathogen of concern.

### DETAILED DESCRIPTION OF THE INVENTION

Provided herein are methods and compositions related to the in vitro production of cell-based products for consumption comprising proteins from exotic, endangered, and extinct species.

Before describing particular embodiments in detail, it is to be understood that the disclosure is not limited to the particular embodiments described herein, which can vary. It is also to be understood that the terminology used herein is for the purpose of describing particular illustrative embodiments only, and is not intended to be limiting unless otherwise defined. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the invention and how to make and use them. The scope and meaning of any use of a term will be apparent from the specific context in which the term is used. As such, the definitions set forth herein are intended to provide illustrative guidance in ascertaining particular embodiments of the invention, without limitation to particular compositions or biological systems.

As used in the present disclosure and the appended claims, the singular forms “a,” “an” and “the” include plural references unless the content clearly dictates otherwise.

Throughout the present disclosure and the appended claims, unless the context requires otherwise, the word “comprise”, or variations such as “comprises” or “comprising”, will be understood to imply the inclusion of a stated element or group of elements but not the exclusion of any other element or group of elements.

Unless specific definitions are provided, the nomenclature utilized in connection with, and the laboratory procedures and techniques of, molecular biology, cell biology, analytical chemistry, and synthetic organic chemistry described herein are those well-known and commonly used in the art. Standard techniques may be used for recombinant technology, molecular biological, microbiological, chemical syntheses, and chemical analyses.

Provided herein are libraries or stockpiles of data that accelerate responses to imminent pandemics and methods of preparing such libraries or stockpiles. Specifically, these libraries or stockpiles enable rapid production of vaccines that target pathogens with pandemic potential.

In some embodiments, methods of the disclosure may entail preparing a library of vaccine data by assessing one or more circulating pathogens of concern; examining whether the circulating pathogens of concern have one or more family members with pandemic potential; pursuant to the assessment and examination steps, identifying a prototype pathogen from at least one of the family members with pandemic potential; creating a vaccine designed to generate an immune response against the prototype pathogen; and collecting data regarding the pathogen and vaccine.

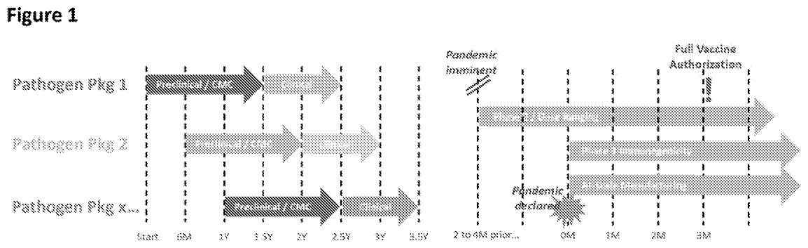

As depicted in FIG. 1, the disclosed method may entail initially compiling preclinical and chemistry, manufacturing, and controls (CMC) data and, subsequently, clinical data in a first set of data related to a first prototype pathogen. In some embodiments, the data may comprise vaccine candidates that target the prototype pathogen. Shortly after, as shown in FIG. 1, as data collection for the first set of data is ongoing, preparation may comprise collecting preclinical, CMC, and clinical data in a second set of data linked to a second prototype pathogen. By way of example, the timespan between identification of the first and second prototype pathogens—and, accordingly, initiation of the first and second sets of data—may be six months. Alternatively, the timespan between identification and initiation may be one year, 1.5 years, two years, or any suitable period. The present disclosure allows for one, two, three, four, five, or any suitable number of datasets at a given time. In some embodiments, several datasets directed to several prototype pathogens may be ongoing at a given time.

In particular embodiments, the data may include data related to any one of preclinical trials, toxicology, chemistry, controls, phase I clinical trials for vaccine candidates that target the prototype pathogen, good laboratory practice, safety, or manufacturing. In preferred embodiments, the data may comprise all data necessary to initiate phase II clinical trials for vaccine candidates.

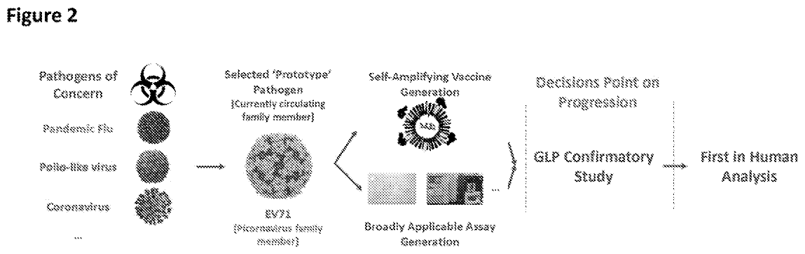

In some embodiments, the library of data related to a prototype pathogen may accelerate future pandemic responses by comprising a package of assays related to either the prototype pathogen, a vaccine that targets the prototype pathogen, or both. By way of example, the assays may comprise pathogen-agnostic assays and pathogen packages that pre-address regulatory agency concerns. For example, as shown in FIG. 2, preparation may first entail assessing circulating pathogens of concern (i.e., influenza virus, poliovirus, or coronavirus) by examining a number of criteria, including whether such circulating pathogens have family members with pandemic potential. Following such review, a prototype pathogen is selected based on circulating family members, similar types of pathogens, related animal strains, and/or constructs generated by examining related pathogenic sequences and understanding the pathogenic target. Lastly, confirmatory and human studies are performed to confirm efficacy and safety.

In certain embodiments, the disclosed method may be repeated for multiple prototype pathogens. By way of example, more than one prototype pathogen may be selected in a given season. For example, at least two, three, four, or five prototype pathogens may be selected in a given season. In particular embodiments, any suitable number of prototype pathogens may be selected in a given season. Selection of prototype pathogens may repeat in subsequent seasons, demonstrating that the disclosed library of data is frequently expanding. In some embodiments, the prototype pathogens under examination may be from related or unrelated families.

In some embodiments, the circulating pathogens of concern may be determined or identified by experts in the field. By way of example, circulating pathogens of concern may be determined or identified based on pathogens of animal source that have crossed over to humans, ability to mutate, or disease burden, in particular if such burden is associated with the potential to initiate a pandemic. In some embodiments, family members of circulating pathogens of concern may also be examined. Alternatively, circulating pathogens of concern may be determined based on transmission potential; ability to evade a host's immune system; particle load; dose infectivity; and capability of crowding, promiscuity, co-infectivity, and/or morbidity; contagiousness during an incubation period; contagiousness prior to development of symptoms or when infected hosts demonstrate only mild symptoms; specific host population factors (i.e. the degree of immunological naivete in a host population); or additional intrinsic microbial pathogenicity characteristics. Transmission potential may include respiratory transmission or transmissibility by skin contact, bodily fluids, airborne particles, contact with feces, and touching of a surface previously touched by an infected individual.

As illustrated in FIG. 1, such libraries enable accelerated responses to pandemics. Specifically, if a pandemic is deemed imminent and is linked to a pathogen with pandemic potential for which a library of data already exists, vaccine manufacturers may turn to that library of data to accelerate production of vaccines in response to the imminent pandemic. In some embodiments, the library of data may accelerate clinical trials for a vaccine candidate. In particular, the library of data may enable immediate initiation of phase II clinical trials for the vaccine candidate, thus preventing any unnecessary and potentially harmful delay in response to the imminent pandemic. By way of example, library data related to the prototype pathogen and the vaccine against that prototype pathogen may serve as a starting point for immediate entry into phase II clinical trials of a vaccine candidate that targets the pathogen that caused the pandemic. Once the pandemic is declared, phase III clinical trials for the vaccine candidate may be initiated, along with at scale manufacturing of the vaccine. In exemplary embodiments, this library of data may accelerate full authorization of the vaccine upon declaration of the pandemic as opposed to merely emergency use authorization (such as, for example, in the COVID-19 pandemic scenario where vaccines against COVID-19 were only authorized for emergency use because of alack of data prior to initiation of vaccine trials). By way of example, the vaccine may be fully authorized for use in as little as three months following declaration of the pandemic. In particular embodiments, the vaccine may be fully authorized for use in a period shorter than three months, for example, two months or even one month. In certain embodiments, manufacturing data related to the prototype pathogen collected prior to the pandemic may advantageously accelerate manufacture of the pandemic vaccine once the pandemic is declared. In some embodiments, the prototype pathogen may be a virus, parasite, bacterium, or fungus. In particular embodiments, the virus may be influenza virus, poliovirus, or coronavirus. In some embodiments, the vaccine may be a DNA-based, RNA-based, protein-based, or vector-based vaccine. For example, the vector-based vaccine may be an adenovirus vector vaccine.

In alternate embodiments, access to a library of data may be offered to a user, for example, by payment of a subscription. In some embodiments, the user may be a vaccine manufacturer. In some embodiments, the user may purchase data or a vaccine candidate of interest from the available library. In particular embodiments, the user may purchase the data or vaccine candidate in anticipation of an upcoming pandemic. In certain embodiments, the user may be a country or countries where pandemics are imminent.

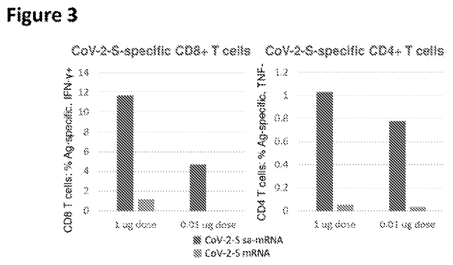

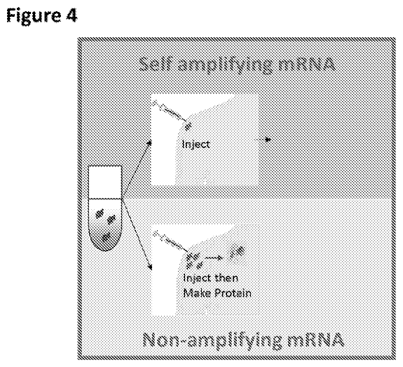

In some embodiments, pandemic vaccines may comprise a self-amplifying mRNA technology. The self-amplifying mRNA technology may provide for vaccines that generate stronger immune responses against pathogens than conventional, non-amplifying mRNA vaccines and protein-based vaccines. In particular, the self-amplifying mRNA technology may generate vaccines that elicit stronger T cell responses, including more robust CD4⁺ and CD8⁺ T cell responses. In particular embodiments, the T cell responses elicited by the self-amplifying mRNA technology may be 5-8 times stronger than that generated by conventional mRNA vaccines, as shown in FIG. 3 comparing the vaccination response against COVID-19. As can be seen in FIG. 3, vaccination with 0.01 μg or 1 μg doses of self-amplifying mRNA (blue) elicited significantly more robust CD4⁺ and CD8⁺ T cells responses compared to vaccination with non-amplifying mRNA (orange). In some embodiments, as shown in FIG. 4, injection of self-amplifying mRNA into patients allows for generation of significantly more mRNA and protein than injection of non-amplifying RNA, which explains, in part, the significant increase in immune response associated with vaccines that include the amplifying technology.

In certain embodiments, vaccines that comprise the self-amplifying mRNA technology may include internal genes.

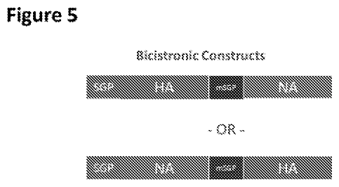

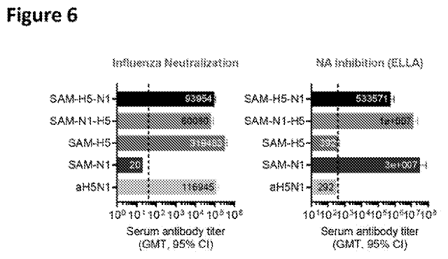

In some embodiments, constructs that comprise the self-amplifying mRNA technology may generate greater breadth by including a wide range of pathogenic antigens. By way of example, as shown in FIG. 5, self-amplifying mRNA bicistronic constructs targeting influenza virus may comprise such antigens as neuraminidase and hemagglutinin, as well as more conserved influenza antigens. Surprisingly, irrespective of the position of neuraminidase and hemagglutinin in such constructs, they both elicit comparably strong responses against influenza in mice. Specifically, as can be seen in FIG. 6, antibody titers against hemagglutinin (H5), neuraminidase (N1), and H5N1 were high, demonstrating strong neutralization and inhibition of influenza virus in mice. Key to the effectiveness of such constructs was the linker protein.

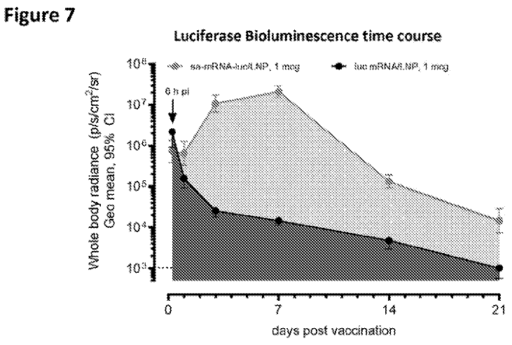

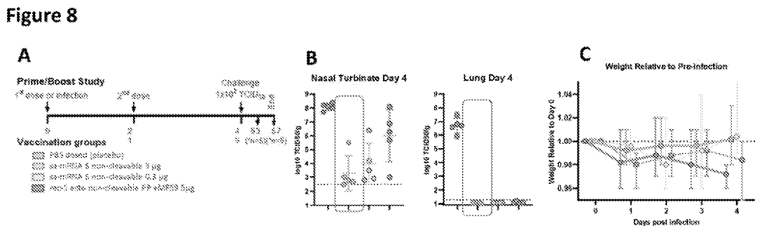

In some embodiments, vaccines comprising the self-amplifying mRNA technology may allow for a smaller number of doses than conventional, non-amplifying mRNA vaccines. As can be seen in FIG. 7, expression of self-amplifying mRNA (red) was prolonged relative to non-amplifying mRNA (black) and even underwent amplification in vivo for seven days, thus explaining how self-amplifying mRNA may be tied to lower dosage. Surprisingly, as shown in FIG. 8, hamsters administered 0.3 μg or 3 μg doses of self-amplifying mRNA vaccines exhibited significant protection when challenged with COVID-19 relative to placebo-treated hamsters or hamsters treated with non-amplifying mRNA plus MF59 adjuvant, further supporting the notion that lower dosing may be possible for self-amplifying mRNA vaccines. In particular, as shown in FIGS. 8B and 8C, viral load was significantly decreased in both the nasal turbinates and lungs of COVID-19-challenged hamsters administered either dose and weight in such hamsters remained largely stable post-infection. Smaller dosage may reduce potential reactogenicity against components of the self-amplifying mRNA technology, such as RNA or lipids, thereby enhancing the safety of such vaccines. In addition, reduced dosage in vaccines comprising the self-amplifying mRNA technology may enable a vaccine dose-sparing strategy that allows for broader population coverage, for example, in the context of pandemics.

In some embodiments, vaccines comprising the self-amplifying mRNA technology may allow for a shorter lead time to adjust the vaccine based on antigenic changes by circulating pathogens. For example, unlike protein, self-amplifying mRNA may be updated more readily following antigen change.

In other embodiments, the self-amplifying mRNA technology may provide an improved response speed to pathogens that could become problematic. In particular, the self-amplifying mRNA technology may increase efficacy against such pathogens via a novel mechanism of action and, additionally, may enable full at-scale vaccine production and delivery.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### Example 1: Accelerated Development of a Coronavirus Pandemic Vaccine

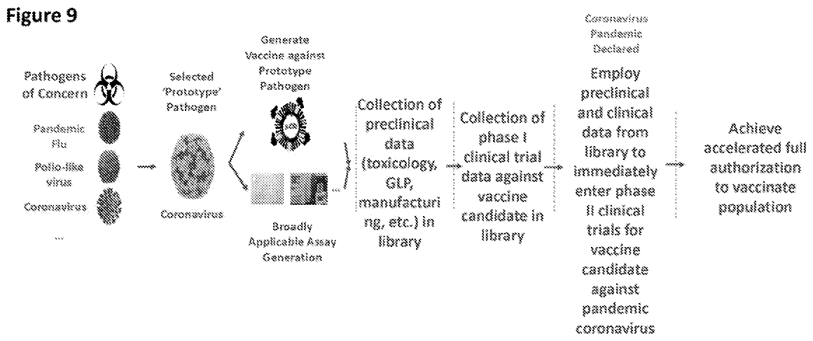

An example of the present disclosure is depicted in the flowchart of FIG. 9 and described herein. Briefly, the example entails first identifying circulating pathogens of concern. In this example, a concerning circulating coronavirus is first identified. A prototype of that circulating coronavirus is next generated based on, for example, related family members of the coronavirus. Next, assays are employed in order to generate a vaccine candidate against the prototype coronavirus. A library of preclinical and clinical data related to the prototype coronavirus and vaccine candidate are collected. Preclinical data includes toxicology, GLP, and manufacturing data, and clinical data includes data related to phase I clinical trials.

While the library of coronavirus data is being generated, a second concerning circulating pathogen is identified, this time an H1N1 influenza virus. Similar to with coronavirus, a prototype of that influenza virus is generated and a library comprising preclinical and clinical data on a vaccine against that influenza virus collected. This process is repeated with a third concerning circulating pathogen, this time EV71. This process is repeated for additional concerning circulating pathogens.

As data on various concerning circulating pathogens is collected, a pandemic against coronavirus is declared imminent. Users, such as countries where the pandemic is imminent or vaccine manufacturers, then gain access to the library of data related to the prototype coronavirus and generate a vaccine candidate against the pandemic coronavirus. Thanks in large part to the library of data on the prototype coronavirus, the pandemic vaccine candidate immediately enters phase II clinical trials. Shortly thereafter (i.e. 1-2 months), the pandemic vaccine candidate progresses to phase III clinical trials and at-scale manufacturing of the vaccine candidate is initiated. A short period later (i.e. three months), the vaccine candidate is fully approved for vaccination of the pandemic-susceptible population.

All of the claims in the claim listing are herein incorporated by reference into the specification in their entireties as additional embodiments.

1. A method of preparing a library of vaccine data comprising:
assessing one or more circulating pathogens of concern;
examining whether the circulating pathogens of concern have one or more family members with pandemic potential;
identifying a first prototype pathogen from at least one of the family members with pandemic potential based on the assessment and examination steps;
creating a first vaccine designed to generate an immune response against the first prototype pathogen;
collecting data regarding the first vaccine;
repeating the method for a second prototype pathogen with pandemic potential and collecting data regarding the second vaccine; and
creating the library of vaccine data from at least the data regarding the first and second vaccines.

2. A method of providing access to a library of vaccine data to a user comprising:
assessing one or more circulating pathogens of concern;
examining whether the circulating pathogens of concern have one or more family members with pandemic potential;
identifying a first prototype pathogen from at least one of the family members with pandemic potential based on the assessment and examination steps;
creating a first vaccine designed to generate an immune response against the first prototype pathogen;
collecting data regarding the first vaccine;
repeating the method for a second prototype pathogen with pandemic potential and collecting data regarding the second vaccine; and
creating the library of vaccine data from at least the data regarding the first and second vaccines;
wherein the user pays for access to the library by subscription.

3. A method of preparing a vaccine for a pathogen of interest comprising choosing a prototype pathogen from a library of vaccine data that is related to the pathogen of interest, wherein the library of vaccine data was prepared by:
assessing one or more circulating pathogens of concern;
examining whether the circulating pathogens of concern have one or more family members with pandemic potential;
identifying a first prototype pathogen from at least one of the family members with pandemic potential based on the assessment and examination steps;
creating a first vaccine designed to generate an immune response against the first prototype pathogen;
collecting data regarding the first vaccine; and
repeating the method for a second prototype pathogen with pandemic potential and collecting data regarding the second vaccine;
creating the library of vaccine data from at least the data regarding the first and second vaccines; and
preparing the vaccine for the pathogen of interest utilizing the data on the vaccines for the first or second prototype pathogens.

4. The method of claim 3, wherein the vaccine for the pathogen of interest is entered into phase II clinical trials.

5. A method of preparing a vaccine of interest for a user comprising choosing a prototype pathogen from a library of vaccine data that is related to a pathogen of interest, wherein the library of vaccine data was prepared by:
assessing one or more circulating pathogens of concern;
examining whether the circulating pathogens of concern have one or more family members with pandemic potential;
identifying a first prototype pathogen from at least one of the family members with pandemic potential based on the assessment and examination steps;
creating a first vaccine designed to generate an immune response against the first prototype pathogen;
collecting data regarding the first vaccine; and
repeating the method for a second prototype pathogen with pandemic potential and collecting data regarding the second vaccine;
creating the library of vaccine data from at least the data regarding the first and second vaccines; and
preparing the vaccine of interest utilizing the data on the vaccines for the first or second prototype pathogens; wherein
the user pays for access to the vaccine of interest by subscription.

6. The method of claim 2, wherein the user selects a vaccine for further development from the library.

7. A method of developing a pandemic vaccine comprising:
identifying a pandemic pathogen;
identifying the prototype pathogen from any of the previous claims that is related to the pandemic pathogen; and
using the vaccine data for the prototype pathogen from the library of vaccine data to develop the pandemic vaccine.

8. The method of claim 1 wherein the pathogens of pandemic potential are chosen by a panel of experts.

9. The method of claim 1, wherein a user has access to the vaccine library data.

10. The method of claim 9, wherein the user pays a subscription fee for access to the vaccine library data.

11. The method of claim 1, wherein a vaccine is chosen for phase 2 clinical trials.

12. The method of claim 1, wherein the pathogens of concern are chosen based on a pathogen of animal source that has crossed over to human.

13. The method of claim 1, wherein the pathogens of concern are chosen based on at least one factor chosen from ability to mutate and a current known disease burden of the pathogens of concern or that of related family members identified as having pandemic potential.

14. The method of claim 1, wherein the pathogens of concern are evaluated based on at least one characteristic chosen from a respiratory mode of transmission; degree of contagiousness during an incubation period, prior to symptom development, and/or when infected individuals show only mild symptoms; specific host population factors (e.g., immunologically naïve persons), and additional intrinsic microbial pathogenicity characteristics.

15. The method of claim 1, wherein the pathogens of concern are evaluated based on at least one transmissibility characteristic, including transmissibility by skin contact, bodily fluids, airborne particles, contact with feces, and touching of a surface previously touched by an infected individual.

16. The method of claim 1, wherein the pathogens of concern are evaluated based on at least one characteristic chosen from transmission potential; ability to evade a host's immune system; particle load; dose infectivity; and capability of crowding, promiscuity, co-infectivity, and/or morbidity.

17. The method of claim 1, wherein the first and second prototype pathogens are viruses, bacteria, parasites, or fungi.

18. The method of claim 1, wherein the vaccine is DNA-based, RNA-based, protein-based, or vector-based.

19. The method of claim 1, wherein the vaccine comprises self-amplifying mRNA technology.

20. The method of claim 1, wherein the collected data comprises preclinical data, toxicology data, chemistry data, GLP and/or GMP manufacturing data, control data, and clinical trial data.

21. A library of vaccines prepared by the method of claim 1.
